(19) United States
(12) Patent Application Publication
(10) Pub. No.: US 2022/0331290 A1
(43) Pub. Date: Oct. 20, 2022

(54) PHARMACEUTICAL COMPOSITION FOR PREVENTING OR TREATING SARCOPENIA, CONTAINING UNNATURAL AMINO ACID

(71) Applicant: MYO-TEC-SCI, Seoul (KR)

(72) Inventors: Hyeon Soo KIM, Seoul (KR); Jae Ho SHIM, Seoul (KR)

(21) Appl. No.: 17/763,597

(22) PCT Filed: Sep. 24, 2020
(86) PCT No.: PCT/KR2020/012984

§ 371 Date: Mar. 24, 2022

(30) Foreign Application Priority Data
Sep. 24, 2019 (KR) 10-2019-0117225
Sep. 23, 2020 (KR) 10-2020-0123054

(51) Int. Cl. A61K 31/401; A61P 21/06; A61K 31/198

(52) U.S. Cl. CPC A61K 31/401; A61P 21/06; A61K 31/198

(57) ABSTRACT

According to one embodiment of the present invention, provided is a composition for preventing or treating sarcopenia, containing, as an active ingredient, at least one type of unnatural amino acid selected from the group consisting of D-leucine, D-alanine and D-proline.

### CROSS-REFERENCE TO RELATED APPLICATIONS

This application is a U.S. national stage entry of International Application No. PCT/KR2020/012984, filed Sep. 24, 2020, which claims priority to Korean Application No. 10-2019-0117225, filed Sep. 24, 2019, and Korean Application No. 10-2020-0123054, filed Sep. 23, 2020, the entire disclosures of which are incorporated herein by reference

### TECHNICAL FIELD

The present disclosure relates to a pharmaceutical composition for preventing or treating sarcopenia containing the unnatural amino acids D-leucine, D-alanine, and D-proline as active ingredients.

### BACKGROUND

The concept of sarcopenia started when Irwin Rosenberg introduced the term “sarcopenia” in 1989. Upon reviewing its origin in Greek, it is a compound word of “sarco” meaning muscle and “penia” meaning reduced. Sarcopenia refers to a decrease in muscle strength due to a decrease in muscle mass associated with aging. Here, “muscle” refers to skeletal muscle and is unrelated to smooth muscle. In other words, sarcopenia refers to the loss of skeletal muscle mass mainly distributed in the extremities, and is distinguished from cachexia, which is a state of significant muscle loss in the late stages of a malignant tumor, muscle wasting due to an acute disease such as influenza, or a disease of the muscle itself (primary muscle disease).

Recently, the prevalence of osteoporosis and sarcopenia is also increasing rapidly as the age group of the elderly 65 years or older has rapidly increased. It is estimated that the gradual decrease in muscle mass occurs after the age of 40 and decreases by 8% every 10 years until the age of 70. It is known that thereafter, even more rapid decreases occur, which can occur by as much as 15% every 10 years. Many follow-up studies have shown that the physiological changes that occur in the elderly are diverse, and in general, muscle mass and bone density decrease simultaneously with increasing age.

There are three major treatment methods for sarcopenia. The first is exercise. It has been reported that exercise increases the protein synthesis ability of skeletal muscle in the short term, and increases muscle strength or motility of the elderly. However, it is not suitable for long-term treatment. Second, testosterone or anabolic steroid can be used as drug treatment, but this induces masculinization in women, and in men, it exhibits side effects such as prostate symptoms. Other approved prescriptions include DHEA (dehydroepiandrosterone) and growth hormone, and studies have reported that it can be used as a treatment method for sites that contain SARMs (selective androgen receptor modulators). In addition, although diet is known as a treatment method, nutritional evaluation shows that malnutrition or modern eating habits are inadequate to maintain adequate total body mass.

Myostatin is a polypeptide growth factor belonging to the superfamily of TGF-β. TGF-β has a large amount of isoforms, which are known to be involved in cell proliferation, apoptosis, differentiation, and bone formation and maintenance (Massague & Chen, 2000). Myostatin belongs to growth differentiation factor (GDF) number 8 among them, is involved in tissue growth and development, and works by activating the Smad signaling system. In addition, it has been reported that the p21 gene inhibits cell cycle and progenitor cell proliferation, thereby affecting bone formation and regeneration. It is known that myostatin is mainly produced in skeletal muscle cells and causes muscle loss and muscle strength reduction in an autocrine manner, and that by inhibiting the expression of IGF-1 or Follistatin involved in muscle hypertrophy, protein synthesis and cell proliferation in myoblast are inhibited.

### SUMMARY

Under this background, the present inventors made intensive efforts to discover a substance capable of treating sarcopenia by inhibiting myostatin expression and promoter activity, which causes muscle loss and muscle strength reduction, and as a result, found that the unnatural amino acids D-leucine, D-alanine, and D-proline could be used for preventing or treating sarcopenia by inhibiting the increase in mRNA expression and the production of myostatin protein, and then completed the present disclosure.

It is an aspect of the present disclosure to provide a pharmaceutical composition for preventing or treating sarcopenia containing an unnatural amino acid as an active ingredient. However, the aspects of the present disclosure are not limited to those mentioned above, and other aspects not mentioned herein will be clearly understood by those skilled in the art from the following description.

According to an example embodiment of the present disclosure, there is provided a pharmaceutical composition for preventing or treating sarcopenia containing, as an active ingredient, at least one type of unnatural amino acid selected from the group consisting of D-leucine, D-alanine, and D-proline.

According to an aspect, the pharmaceutical composition may inhibit myostatin mRNA or protein expression.

According to an aspect, the pharmaceutical composition may reduce a myostatin promoter activity.

According to an aspect, the pharmaceutical composition for preventing or treating sarcopenia may contain 0.001 mM to 10 mM of the unnatural amino acid.

According to an example embodiment of the present disclosure, there is provided a preparation for preventing or treating sarcopenia including a pharmaceutical composition containing, as an active ingredient, at least one type of unnatural amino acid selected from the group consisting of D-leucine, D-alanine, and D-proline, and at least one selected from the group consisting of a pharmaceutically acceptable carrier, an excipient, a diluent, a stabilizer, and a preservative.

According to an aspect, the preparation may have a formulation of powder, granule, tablet, capsule or injection.

### Advantageous Effects

The pharmaceutical composition of the present disclosure for preventing or treating sarcopenia containing an unnatural amino acid as an active ingredient inhibits the increase in myostatin protein production and mRNA expression, which directly affects muscle loss and muscle strength reduction, and thus can exhibit a more fundamental preventive or therapeutic effect of sarcopenia.

It should be understood that the effects of the present disclosure are not particularly limited to those described above, and the present disclosure includes all effects that can be deduced from the detailed description of the invention or the configurations of the invention described in the claims.

### BRIEF DESCRIPTION OF DRAWINGS

FIG. 1 illustrates a comparison of myostatin promoter activities of mouse skeletal muscle cells C2C12 in a complete medium containing L-leucine and a medium without L-leucine.

FIG. 2 illustrates a comparison of a myostatin promoter activity of mouse skeletal muscle cells C2C12 after treatment with D-leucine at a concentration of 0.001-1 mM in a medium without L-leucine.

FIG. 3 illustrates a comparison of a myostatin promoter activity of mouse skeletal muscle cells C2C12 after treatment with D-leucine and a mixture of D-leucine at a concentration of 0.001-1 mM in a medium without L-leucine.

FIG. 4 illustrates a comparison of myostatin mRNA expression levels of mouse skeletal muscle cells C2C12 after treatment with D-leucine in a complete medium containing L-leucine and a medium without L-leucine.

FIG. 5 illustrates a comparison of myostatin mRNA expression levels of mouse skeletal muscle cells C2C12 after treatment with D-leucine at a concentration of 0.001-1 mM in a medium without L-leucine.

FIG. 6 illustrates a comparison of a myostatin promoter activity of mouse skeletal muscle cells C2C12 after treatment with D-alanine at a concentration of 0.01-10 mM in a medium without L-leucine for 12 hours.

FIG. 7 illustrates a comparison of mRNA expression levels of mouse skeletal muscle cells C2C12 after treatment with D-alanine at a concentration of 0.001-1 mM in a medium without L-leucine for 12 hours.

FIG. 8 illustrates a comparison of a myostatin promoter activity of mouse skeletal muscle cells C2C12 after treatment with D-proline at a concentration of 0.001-1 mM in a medium without L-leucine for 24 hours.

FIG. 9 illustrates a comparison of mRNA expression levels of mouse skeletal muscle cells C2C12 after treatment with D-proline at a concentration of 0.1-1 mM in a medium without L-leucine for 24 hours.

FIG. 10 illustrates a comparison of a myostatin promoter activity of mouse skeletal muscle cells C2C12 after treatment with 9 other amino acids in a medium without L-leucine.

FIG. 11 illustrates a comparison of a myostatin promoter activity of mouse skeletal muscle cells C2C12 after treatment with D-tryptophan in a medium without L-leucine.

### DETAILED DESCRIPTION

Hereinafter, example embodiments will be described in detail with reference to the accompanying drawings. However, since various changes may be made to the example embodiments, the scope of right of the patent application is not limited or restricted by these example embodiments. It should be understood that all modifications, equivalents and substitutes for the example embodiments are included within the scope of right.

The terms used in the example embodiments are used for description purposes only and should not be construed as being limited by these example embodiments. The terms in singular form may include plural forms unless otherwise specified. It will be understood that the terms “comprising” or “having,” when used herein, specify the presence of stated features, integers, steps, operations, elements, components, or combinations thereof, but do not preclude the presence or addition of one or more other features, integers, steps, operations, elements, components, or combinations thereof.

Unless otherwise defined, all technical and scientific terms used in the example embodiments have the same meanings as commonly understood by those skilled in the technical field to which the example embodiments pertain. It will be further understood that terms, such as those defined in commonly used dictionaries, should be interpreted as having a meaning that is consistent with their meanings of the context of the relevant art and the present disclosure, and will not be interpreted in an idealized or overly formal sense unless expressly so defined herein.

In addition, in the description with reference to the accompanying drawings, the same reference numerals are assigned to the same components regardless of the reference numerals, and redundant descriptions thereof will be omitted. In describing the example embodiments, when it is determined that a detailed description of related known technologies may unnecessarily obscure the gist of the example embodiments, the detailed description thereof will be omitted.

According to an example embodiment of the present disclosure, there is provided a pharmaceutical composition for preventing or treating sarcopenia containing, as an active ingredient, at least one type of unnatural amino acid selected from the group consisting of D-leucine, D-alanine, and D-proline.

As used herein, the term “prevention” means all actions for inhibiting or delaying the onset of sarcopenia by administration of a composition. As used herein, the term “treatment” means all actions involved in alleviating or beneficially changing symptoms of sarcopenia by administration of a composition.

The pharmaceutical composition may inhibit myostatin mRNA or protein expression, and may reduce a myostatin promoter activity, so that it is possible to more fundamentally prevent and treat muscle loss and muscle strength reduction.

The pharmaceutical composition for preventing or treating sarcopenia according to an example embodiment of the present disclosure may contain the unnatural amino acid derivative at a concentration of 0.001 mM or more, specifically, it may contain the same at a concentration of 0.001 mM to 10 mM, and more specifically 0.001 mM to 1 mM.

The pharmaceutical composition of the present disclosure may be prepared by a method known in the pharmaceutical field in order to be used as pharmaceuticals, and is mixed with a pharmaceutically acceptable carrier, an excipient, a diluent, a stabilizer, a preservative, etc. to be prepared and used in a formulation such as powder, granule, tablet, capsule or injection. In addition, the composition may be prepared as a sustained-release preparation so that the release of an active ingredient occurs slowly, including a base used for sustained-release purpose in addition to the active ingredient.

Pharmaceutically acceptable carriers may further include, for example, carriers for oral administration or carriers for parenteral administration. The carriers for oral administration may include lactose, starch, cellulose derivatives, magnesium stearate and stearic acid. In addition, it may include various drug delivery materials used for oral administration to a peptide agent. In addition, the carriers for parenteral administration may include water, suitable oils, saline, aqueous glucose and glycols, and further include stabilizers and preservatives. Suitable stabilizers include antioxidants such as sodium hydrogen sulfite, sodium sulfite or ascorbic acid. Suitable preservatives include benzalkonium chloride, methyl- or propyl-parabens and chlorobutanol. The pharmaceutical composition of the present disclosure may further include a lubricant, a wetting agent, a sweetening agent, a flavoring agent, an emulsifier, a suspending agent, etc. in addition to the above components.

Examples of the diluent include non-aqueous solvents such as propylene glycol, polyethylene glycol, and vegetable oil such as olive oil and peanut oil, and aqueous solvents such as salt water (preferably 0.8% of salt water) and water including a buffered medium (preferably 0.05 M of phosphate buffer), but are not limited thereto.

Examples of the excipient include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol, and the like, but are not limited thereto.

Examples of the stabilizer include carbohydrates such as sorbitol, mannitol, starch, sucrose, dextran, glutamate, and glucose, or proteins such as animal, vegetable, or microbial proteins such as milk powder, serum albumin, casein and the like, but are not limited thereto.

Examples of the preservative may include thimerosal, merthiolate, gentamicin, neomycin, nystatin, amphotericin B, tetracycline, penicillin, streptomycin, polymyxin B and the like, but are not limited thereto.

The pharmaceutical composition of the present disclosure may be administered to a mammal including a human using any method. For example, the pharmaceutical composition may be orally or parenterally administered. A parenteral administration method may include intravenous, intramuscular, intra-arterial, intramedullary, intradural, intracardiac, percutaneous, subcutaneous, intraperitoneal, intranasal, intestinal, local, sublingual or intrarectal administration, but is not limited thereto.

The pharmaceutical composition of the present disclosure may be formulated into preparations for oral or parenteral administration, depending on the route of administration as described above.

The total effective amount of the pharmaceutical composition of the present disclosure may be administered to a patient in a single dose, and may be administered by a fractionated treatment protocol that is administered for a long period of time in multiple doses. The pharmaceutical composition of the present disclosure may vary the content of the active ingredient according to the severity of the disease. The dosage thereof may be determined in consideration of various factors not only preparation methods, routes of administration, and the number of treatments, but also the patient's age, weight, health condition, disease severity, administration time and method. In consideration thereof, those skilled in the pertinent technical field will be able to determine an appropriate effective dosage of the composition of the present disclosure. The pharmaceutical composition according to the present disclosure is not particularly limited in its formulation, route of administration, and method of administration as long as it exhibits the effects of the present disclosure.

Hereinafter, the present disclosure will be described in more detail by way of examples. However, these examples are only for illustrating the present disclosure, and the scope of the present disclosure is not limited to these examples.

### Example 1. Inhibitory Effect of Myostatin Promoter Activity

1) Experiment on Inhibitory Effect of Myostatin Promoter Activity by D-Leucine or DL-Leucine

After seeding 1×10⁵ cells of mouse skeletal muscle cells C2C12 (ATCC, US) in a 12-well culture plate, on the next day, pGL4.15 empty vector and pGL4.15-MSTN vector containing myostatin promoter were transfected using Lipofectamine 2000 for 4 hours. After 4 hours, the complete medium (0.8 mM L-leucine) and the medium without L-leucine were treated with D-leucine or DL-leucine at concentrations of 0.001 mM, 0.01 mM, 0.1 mM, and 1 mM, respectively. After one day, a dual luciferase assay kit (Promega Inc.) was used to measure the promoter activity of myostatin.

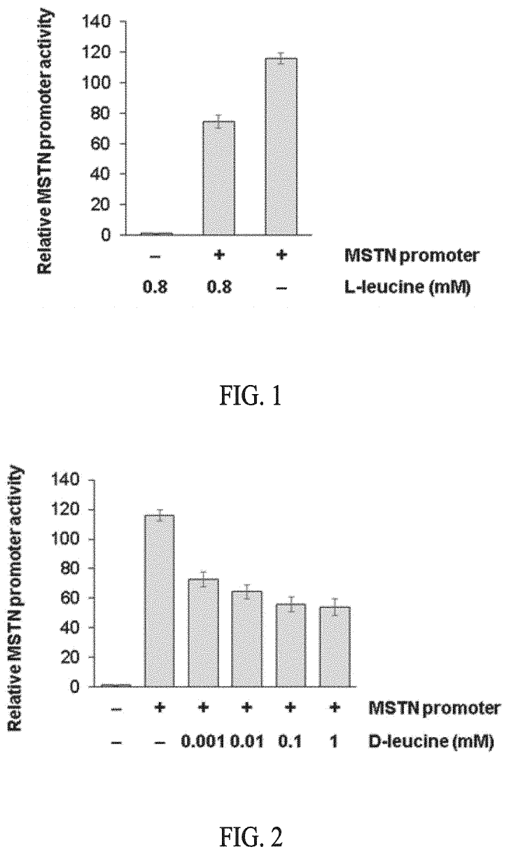

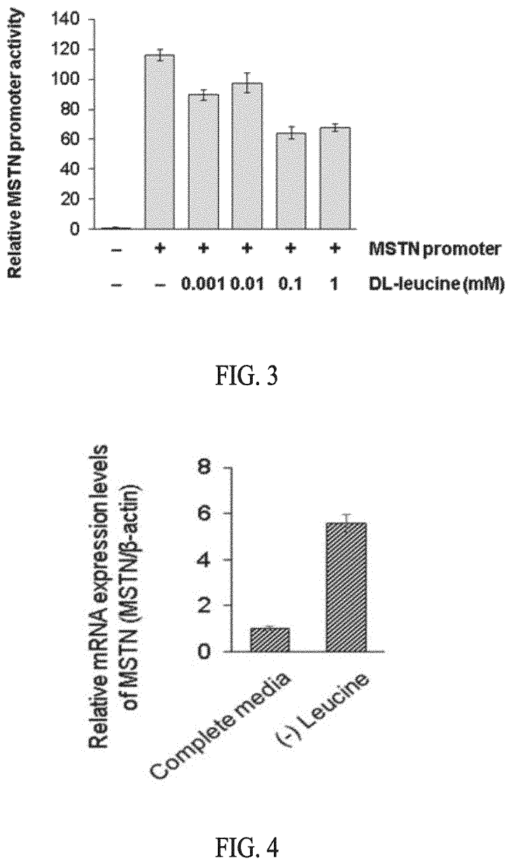

As a result, it was identified that the myostatin promoter activity of C2C12 cells in the medium without L-leucine was increased by 55% compared to the complete medium (see FIG. 1), and that when D-leucine and DL-leucine were treated at concentrations of 0.001 mM, 0.01 mM, 0.1 mM, and 1 mM, respectively, in a medium without L-leucine, the myostatin promoter activity was reduced by 54% when treated with 1 mM D-leucine and by 41% when treated with 1 mM DL-leucine (see FIGS. 2 and 3).

2) Experiment of Inhibitory Effect of Myostatin Promoter Activity by D-Alanine

In the same manner as the experiment to examine the inhibitory effect of a myostatin promoter activity by D-leucine or DL-leucine, D-alanine was treated at concentrations of 0.01 mM, 0.1 mM, 1 mM, and 10 mM in a medium without L-leucine, and a dual luciferase assay kit (Promega Inc.) was used to measure the promoter activity of myostatin.

As a result, it was identified that the promoter activity of myostatin decreased when D-alanine was added at a concentration of 0.01 mM or more (see FIG. 6).

3) Experiment of Inhibitory Effect of Myostatin Promoter Activity by D-Proline

In the same manner as in the experiment to examine the inhibitory effect of a myostatin promoter activity by D-leucine or DL-leucine, and D-alanine, D-proline was treated at concentrations of 0.001 mM, 0.01 mM, 0.1 mM, and 1 mM in a medium without L-leucine, and a dual luciferase assay kit (Promega Inc.) was used to measure the promoter activity of myostatin.

As a result, it was identified that the promoter activity of myostatin decreased when D-proline was added at a concentration of 0.001 mM or more (see FIG. 8).

### Example 2. Inhibitory Effect of Myostatin mRNA Expression

1) Experiment of Inhibitory Effect of Myostatin mRNA Expression by D-Leucine

After seeding mouse skeletal muscle cells C2C12 (ATCC, US) 3×10⁵ cells in a 6-well culture plate, on the next day, D-leucine was treated at concentrations of 0.01 mM, 0.1 mM, and 1 mM in a complete medium (0.8 mM L-leucine) and a medium without L-leucine. After one day, the mRNA expression level of myostatin was identified using qRT-PCR, and the myostatin mRNA expression value was corrected using the expression value of beta-actin.

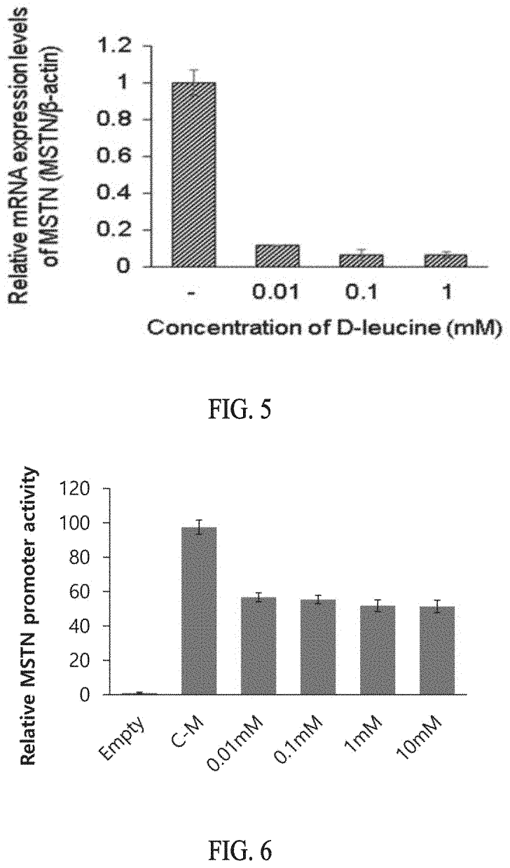

As a result, it was observed that the myostatin mRNA expression of C2C12 cells in the medium without L-leucine was increased 5.5-fold compared to the complete medium containing L-leucine at a concentration of 0.8 mM (see FIG. 4), and in the medium without L-leucine, when D-leucine at concentrations of 0.01 mM, 0.1 mM, and 1 mM was treated, the expression of myostatin mRNA was decreased in a dose-dependent manner (see FIG. 5).

2) Experiment of Inhibitory Effect of Myostatin mRNA Expression by D-Alanine

In the same manner as the experiment to examine the inhibitory effect of myostatin mRNA expression by D-leucine, D-alanine was treated at concentrations of 0.001 mM, 0.01 mM, 0.1 mM, and 1 mM in a medium without L-leucine, the myostatin mRNA expression level was identified using qRT-PCR, and the myostatin mRNA expression value was corrected using the expression value of beta-actin.

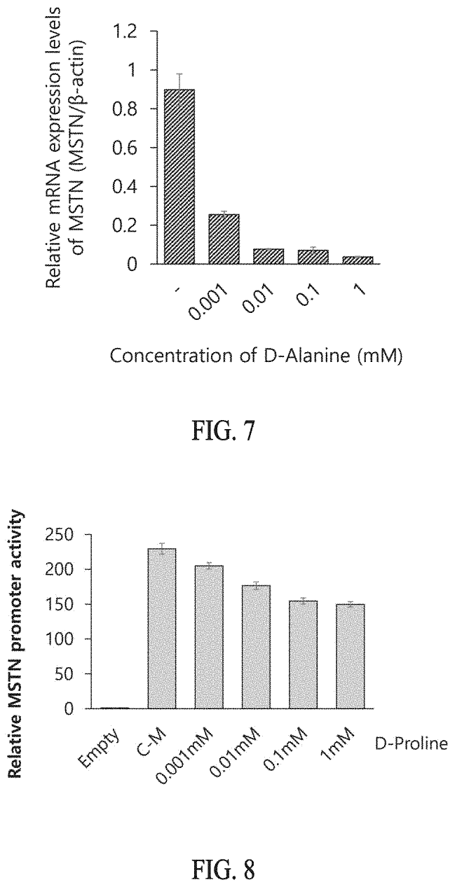

As a result, it was identified that the mRNA expression level of myostatin decreased when D-alanine was added at a concentration of 0.001 mM or more (see FIG. 7).

3) Experiment of inhibitory effect of myostatin mRNA expression by D-proline

In the same manner as the experiment to examine the inhibitory effect of myostatin mRNA expression by D-leucine and D-alanine, D-proline was treated at concentrations of 0.1 mM and 1 mM in a medium without L-leucine, the myostatin mRNA expression level was identified using qRT-PCR, and the myostatin mRNA expression value was corrected using the expression value of beta-actin.

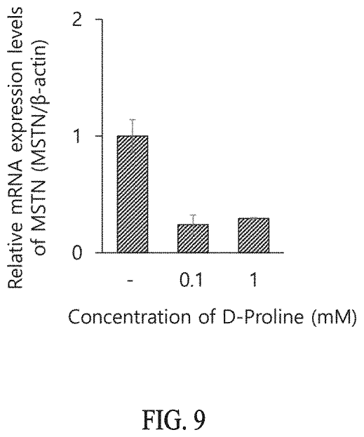

As a result, it was identified that the mRNA expression level of myostatin decreased when D-proline was added at a concentration of 0.1 mM or more (see FIG. 9).

### Comparative Example. Experiment of Inhibitory Effect of Myostatin Promoter Activity by Other 10 Amino Acids

In the same manner as the myostatin promoter activity experimental method performed in Example 1 above, D-phenylalanine (0.001-0.1 mM), D-serine (0.001-0.1 mM), D-tyrosine (0.0001-0.01 mM), D-asparagine monohydrate (0.001-0.1 mM), D-aspartic acid (0.0001-0.01 mM), D-methionine (0.001-0.1 mM), D-cysteine hydrochloride monohydrate (0.001-0.1 mM), D-glutamic acid (0.0001-0.01 mM), D-arginine (0.001-0.1 mM), and D-tryptophan (0.001-0.1 mM) were treated, respectively. The myostatin mRNA expression level was identified using qRT-PCR, and the myostatin mRNA expression value was corrected using the expression value of beta-actin.

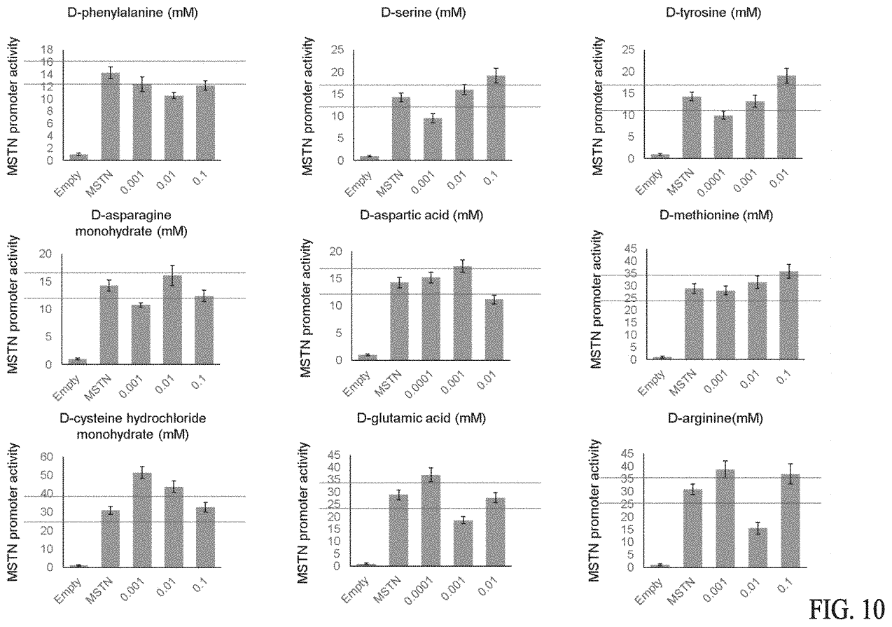

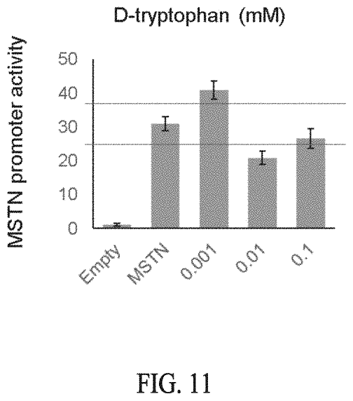

As a result, it was identified that the 10 amino acids did not result in inhibition of a myostatin promoter activity, unlike the treatment with D-leucine, D-alanine, and D-proline (see FIGS. 10 and 11).

The results of the above examples suggest that the unnatural amino acids D-leucine, D-alanine, and D-proline of the present disclosure can be usefully used for the prevention or treatment of sarcopenia by inhibiting myostatin mRNA expression and promoter activity in muscle cells.

Although the example embodiments have been described based on the limited drawings as described above, those skilled in the pertinent technical field may apply various technical modifications and variations based thereon. For example, even when the described technologies are performed in a different order from that in the described method, and/or the described components are coupled or combined in a manner different from that as described above, or are replaced or substituted with other components or equivalents, appropriate results may be achieved.

Therefore, other implementations, other example embodiments, and equivalents to claims also fall within the scope of the following claims.

1. A method for preventing or treating sarcopenia, the method comprising:
administering to a subject—a composition containing, as an active ingredient, at least one type of unnatural amino acid selected from the group consisting of D-leucine, D-alanine, and D-proline.

2. The method of claim 1, wherein the composition inhibits myostatin mRNA or protein expression.

3. The method of claim 1, wherein the composition reduces a myostatin promoter activity.

4. The method of claim 1, wherein the composition contains 0.001 mM to 10 mM of the unnatural amino acid.

5. A preparation for preventing or treating sarcopenia including at least one selected from the group consisting of a pharmaceutically acceptable carrier, an excipient, a diluent, a stabilizer and a preservative, and at least one type of unnatural amino acid selected from the group consisting of D-leucine, D-alanine, and D-proline.

6. The preparation of claim 5, wherein the pharmaceutically acceptable carrier includes carriers for oral administration or carriers for parenteral administration.

7. The preparation of claim 5, wherein the excipient includes at least one selected from the group consisting of starch, glucose, lactose, sucrose, gelatin, malt, rice, flour, chalk, silica gel, sodium stearate, glycerol monostearate, talc, sodium chloride, dried skim milk, glycerol, propylene, glycol, water, and ethanol.

8. The preparation of claim 5, wherein the diluent includes at least one selected from the group consisting of propylene glycol, polyethylene glycol, olive oil, peanut oil, salt water, water, and phosphate buffer.

9. The preparation of claim 5, wherein the preservative includes at least one selected from the group consisting of thimerosal, merthiolate, gentamicin, neomycin, nystatin, amphotericin B, tetracycline, penicillin, streptomycin, and polymyxin B.

10. The preparation of claim 5, wherein the preparation has a formulation of powder, granule, tablet, capsule or injection.

11. The method of claim 1, the method further comprising measuring an expression level of myostatin in muscle cells of the subject before and after the administering.

12. The method of claim 1, the subject is a subject whose muscle strength is weakened and/or muscle loss has progressed, or a subject with a genetic predisposition to sarcopenia.
